(19) United States
(12) Patent Application Publication
(10) Pub. No.: US 2026/0304590 A1
(43) Pub. Date: Oct. 1, 2026

(54) BEAM-MODIFYING DEVICES FOR ULTRA-HIGH DOSE RATE RADIATION THERAPY

(71) Applicant: Ohio State Innovation Foundation, Columbus, OH (US)

(72) Inventors: Charles Allan ZIMMERMAN, Columbus, OH (US); Ahmet Sedat AYAN, New Albany, OH (US); Ashley Joy CETNAR, Grove City, OH (US); Sagarika JAIN, Columbus, OH (US); Nilendu GUPTA, Westerville, OH (US)

(21) Appl. No.: 19/481,042

(22) PCT Filed: May 1, 2024
(86) PCT No.: PCT/US2024/027137

§ 371 Date: Nov. 3, 2025

Related U.S. Application Data
(60) Provisional application No. 63/500,723, filed on May 8, 2023.

(51) Int. Cl. H05H 7/12; A61N 5/10; G21K 1/10

(52) U.S. Cl. CPC H05H 7/12; A61N 5/10; G21K 1/10; A61N 2005/1095; G21K 2201/061; G21K 2201/067; G21K 2201/068; H05H 2007/125; H05H 2277/11

(57) ABSTRACT

A beam-modifying assembly for an ultra-high dose rate (UHDR) electron linear accelerator that generally includes a beam modifier formed of a first material that defines a first layer and a second material that defines a second layer, the second material being different from the first material, wherein the first layer is provided on top of the second layer such that a first surface of the beam modifier comprises the first material and a second surface of the beam modifier comprises the second material, the second surface being on an opposite side of the beam modifier from the first surface; and a mounting assembly configured to retain the beam modifier, wherein the mounting assembly is adapted to be installed at an exit window of the linear accelerator such that the beam modifier is positioned at the exit window of the linear accelerator, wherein the first surface of the beam modifier faces the exit window and the second surface faces a subject to be irradiated.

### CROSS-REFERENCE TO RELATED APPLICATION

This application claims the benefit of U.S. Provisional Application 63/500,723, filed May 8, 2023, the contents of which are hereby incorporated in its entirety.

### BACKGROUND

Ultra-high dose rate radiotherapy (UHDR RT) is a growing field of study in the treatment of tumors, where ultra-high dose rate linear accelerators (linacs) are used to irradiate target areas on a subject. Unlike traditional forms of radiotherapy, UHDR RT has been demonstrated to limit trauma to normal tissue around a tumor without losing effectiveness on the tumor itself. Typically, UHDR RT dose rates exceed 40 Gy/sec; thus, the amount of radiation delivered in UHDR RT can be orders of magnitude larger than what is delivered in conventional electron therapy. This is especially true for commissioning and acceptance testing, which involves long beam-on times.

### SUMMARY

One implementation of the present disclosure is a beam-modifying assembly for an ultra-high dose rate (UHDR) electron linear accelerator. The beam-modifying assembly generally includes: a beam modifier formed of a first material that defines a first layer and a second material that defines a second layer, the second material being different from the first material, wherein the first layer is provided on top of the second layer such that a first surface of the beam modifier includes the first material and a second surface of the beam modifier includes the second material, the second surface being on an opposite side of the beam modifier from the first surface; and a mounting assembly configured to retain the beam modifier, wherein the mounting assembly is adapted to be installed at an exit window of the linear accelerator such that the beam modifier is positioned at the exit window of the linear accelerator, wherein the first surface of the beam modifier faces the exit window and the second surface faces a subject to be irradiated.

In some implementations, the first material is a low-atomic-number (low-Z) material and the second material is a high-atomic-number (high-Z) material.

In some implementations, the first material is acrylic.

In some implementations, the second material is copper.

In some implementations, the beam modifier is cylindrical in shape.

In some implementations, the second layer forms a flange, and wherein a base of the flange interfaces with the mounting assembly to retain the beam modifier in the mounting assembly.

In some implementations, the first layer of the beam modifier is formed in a cylindrical shape defined by a first diameter, and wherein the second layer is formed in the shape of a flange having a base diameter that is wider than the first diameter.

In some implementations, the beam-modifying assembly further includes a third layer formed of a third material that is different from the first material and the second material, wherein the third layer is provided on a bottom surface of the second layer such that the second surface of the beam modifier includes the third material.

In some implementations, the third material is a metal having K-shell absorption energies that match characteristic X-rays of the second material.

In some implementations, the beam modifier is 3D printed, cast, or milled from the first material and the second material.

In some implementations, a central opening is formed in the beam modifier, the central opening extending from the first surface to the second surface along a longitudinal axis of the beam modifier.

In some implementations, the central opening is cylindrical in shape.

In some implementations, the central opening is square in shape.

In some implementations, the first layer has a relative thickness that is 50% of a thickness of the second layer.

In some implementations, a beam-modifying assembly, wherein beam modifier further includes a plurality of fins that extend from the first layer and into the second layer, wherein the plurality of fins are formed of the first material.

### BRIEF DESCRIPTION OF THE DRAWINGS

FIG. 1 is a diagram showing a side view of an example ultra-high dose rate (UHDR) linear accelerator (linac) with a beam-modifying device installed, according to some implementations.

FIG. 2 is a diagram showing a front view of the example UHDR-enabled linac with a beam-modifying device installed, according to some implementations.

FIG. 3 is an illustration of a beam-modifying device for UHDR-enabled linacs, according to some implementations.

FIG. 4 is a diagram illustrating the layers of the beam-modifying device of FIG. 3, according to some implementations.

FIG. 5 is a diagram of simulation results for the beam-modifying device of FIG. 3, according to some implementations.

FIGS. 6-8 are various perspective views of a first configuration of the beam-modifying device of FIG. 3, according to some implementations.

FIGS. 9 and 10 are various perspective views of a second configuration of the beam-modifying device of FIG. 3, according to some implementations.

FIG. 11 is a diagram of an example implementation of the beam-modifying device of FIG. 3, according to some implementations.

FIG. 12 is a graph illustrating results of simulating a beam-modifying device formed of a single material, according to some implementations.

FIG. 13 is a graph illustrating results of simulating a beam-modifying device formed of multiple different materials, according to some implementations.

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

### DETAILED DESCRIPTION

Referring generally to the figures, a beam-modifying/forming device for ultra-high dose rate electron radiotherapy (UHDR RT), e.g., for commissioning and acceptance testing, is shown, accordingly to various implementations. Generally, the beam-modifying device disclosed herein is formed of two or more layers each having unique properties/characteristics. In a two-layer configuration, for example, the beam-modifying device can include a first layer of low atomic number (Z) material with a high collision mass stopping power and a second layer of a high-Z material that stops remaining electrons and attenuates Bremsstrahlung. The beam-modifying device is generally sized to fit into the electron applicator cone of a suitable UHDR RT-enabled linear accelerator (linac). In addition, the size, shape, and other characteristics of the beam-modifying device can be selected to create irregular beam fields, customize the beam shape and/or shape of the irradiation field, etc.

As mentioned above, the amount of radiation delivered in UHDR RT commissioning and acceptance testing can be orders of magnitude larger than what is delivered in conventional electron therapy. Bremsstrahlung radiation emitted by electron interactions with high-Z collimation devices can become a significant source of radiation. Thus, a beam-modifying device designed for UHDR RT commissioning would interact with electrons in such a way as to minimize stray Bremsstrahlung radiation. Through the stacking of different materials with desirable properties, the beam-modifying device described herein meets the need of sufficiently blocking the electron beam output by a UHDR RT linac while remaining lightweight and cost-effective.

### Overview

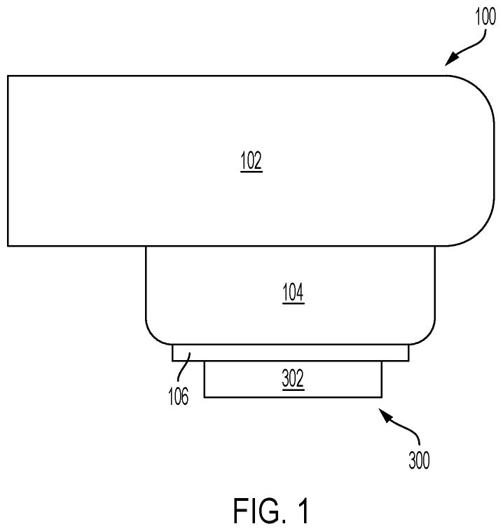

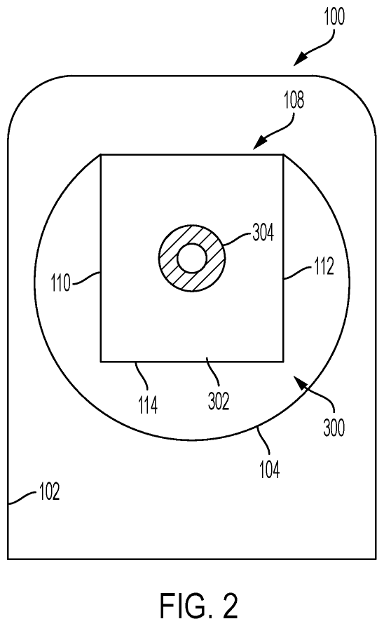

Referring generally to FIGS. 1 and 2, an example linac 100 having a beam-modifying assembly 300 installed thereon is shown from a variety of perspectives. In particular, FIG. 1 shows a side-view of linac 100 and beam-modifying assembly 300 and FIG. 2 shows a bottom-up of linac 100 and beam-modifying assembly 300, according to various implementations. As described herein, linac 100 is generally configured for UHDR RT. In particular, linac 100 is generally a linear accelerator configured to irradiate a target arca of a subject at a dose rate of greater than 40 Gy/sec to achieve the “FLASH” effect for biological sparing of normal tissue. In some implementations, linac 100 is configured to produce an electron beam having an energy of 16 MeV. In one example, linac 100 is a Varian® Clinac™ linac; however, it should be appreciated that linac 100 is not limited in this regard.

As shown, linac 100 generally includes a body 102, also called a gantry, and a treatment head 104. For conciseness, body 102 and treatment head 104, along with operating procedures for linac 100, are not described in detail herein, as they will be readily understood by those of skill in the art. However, it should be understood that treatment head 104 generally includes an exit window for emitting radiation. In some implementations, an accessory rail assembly 106 is used to attach various accessories, such as beam-modifying devices, at or near an exit window of treatment head 104 and is fixed to a bottom side of treatment head 104. As shown in FIG. 2, for example, accessory rail assembly 106 of linac 100 may be generally circular in shape (e.g., when viewed from the bottom) with an opening 108 for inserting accessories. Opening 108 is generally defined by first and second side rails 110, 112 for, for example, retaining beam-modifying assembly 300, and an end wall 114. In some implementations, first and second side rails 110, 112 are L-shaped when viewed from the front.

It should be understood that the accessory rail or accessory mounting points on various linacs may vary in size, shape, arrangement, and configuration, all of which are contemplated herein. It should also be understood that the particular configuration of linac 100 shown in FIGS. 1 and 2 is not intended to be limiting. For example, as described in greater detail below, beam-modifying assembly 300 may be attached to linac 100 through a variety of other means. That is to say, other configurations of linac 100 may have different means of attaching beam-modifying devices and other accessories at or near an exit window. For example, in some implementations, beam-modifying assembly 300 may be removably attached to linac 100 via a frame assembly, bolts or screws, threading, etc. The present disclosure is not intended to be limiting in this regard.

As described in greater detail below, beam-modifying assembly 300 generally includes two main components: a mounting assembly 302 and a beam modifier 304. Mounting assembly 302 is generally configured to retain beam modifier 304 and is used to attach beam modifier 304 to linac 100. As shown in FIG. 2, for example, mounting assembly 302 may be generally square or rectangular in shape, in implementations where linac 100 utilizes accessory rail assembly 106 to retain accessories. In some implementations, the dimensions of mounting assembly 302 are selected based on the configuration of accessory rail assembly 106, or more specifically, based on the size of first and second side rails 110, 112. An example configuration of mounting assembly 302 and/or beam modifier 304 is described in U.S. Provisional Pat. App. No. 63/482,175, filed Jan. 30, 2023, which is incorporated herein by reference in its entirety. As mentioned above, it should be appreciated that in various other implementations, mounting assembly 302 may have a different shape, different dimensions, and/or may attach to linac 100 in another manner.

In the example of FIGS. 1 and 2, beam modifier 304 is shown to be retained by mounting assembly 302 and it thereby attached to linac 100 via mounting assembly 302. Additionally, FIG. 2 shows that, in at least one configuration, beam modifier 304 may be cylindrical in shape; however, it should be appreciated that beam modifier 304 may be other sizes and shapes. As mentioned above, beam modifier 304 is generally configured to modify or “shape” an electron beam emitted by linac 100. Accordingly, beam modifier 304 may be positioned at or near an exit window of linac 100 to modify the beam spectrum/shape as the electron beam exits linac 100. In some implementations, beam-modifying assembly 300 is configured to fits into the electron applicator cone of linac 100. As described in greater detail below, beam modifier 304 can also be custom fabricated to create irregular beam fields, reducing the radiation dose beyond the blocked portion to less than 1% of central axis dose.

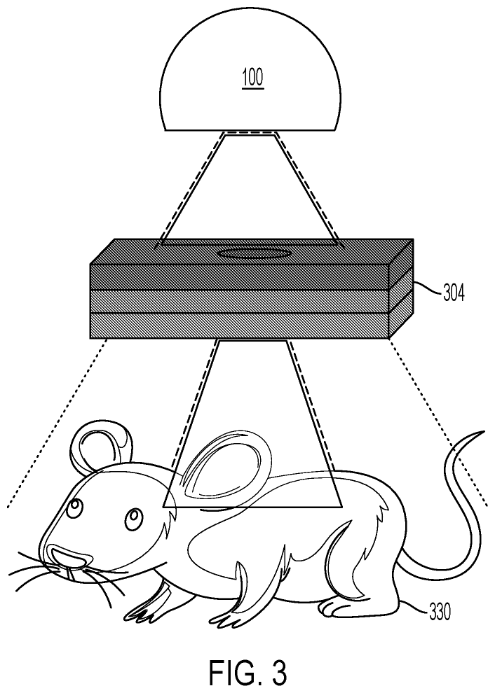

With additional reference to FIG. 3, beam modifier 304 is shown to form the electron beam emitted by linac 100 by, at least in part, blocking the edges of the emitted beam. This focuses the beam onto a target area of a subject 330. In practice, for example, UHDR research often relies on small animal studies. In most of these experiments, test subjects are only exposed to a portion of the field. UHDR systems require commissioning and quality assurance testing for such small-field experiments, which can result in several thousand Gy delivered to isocenter in a few seconds. It is important that the radiation received by operators is kept below regulatory limits during such testing and minimize the total amount of radiation to subjects/patients outside of the treatment field.

### Beam-Modifying Device

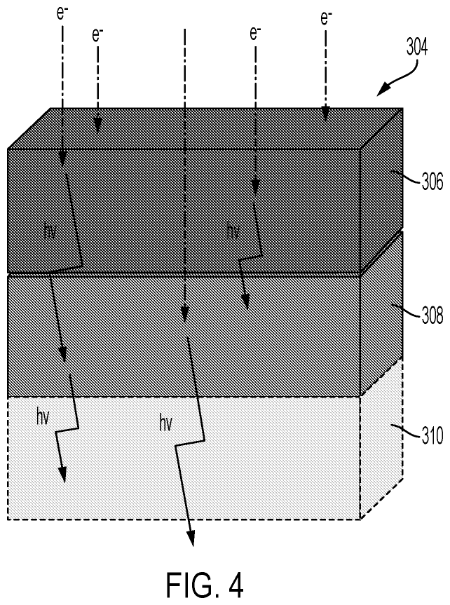

Referring now to FIG. 4, a diagram illustrating the layers of beam modifier 304 is shown, according to some implementations. As described herein, beam modifier 304 is generally a multi-layer structure formed of multiple layers of different materials. As shown in FIG. 4, beam modifier 304 may include at least a first layer 306 and a second layer 308. Each of layers 306, 308 are generally formed of different materials, or rather, materials having different properties. In some implementations, first layer 306 is provided on top of second layer 308 such that a first surface or the “top” side of beam modifier 304 is the first material and a second surface or the “bottom” side of beam modifier 304 is the second material. In other words, a top layer of beam modifier 304 is the first material and a bottom layer of beam modifier 304 is the second material. In some implementations, beam modifier 304 also includes an optional third layer 310 formed of a third material that is different from the first and second materials of layers 306, 308, respectively.

This multilayer design takes advantage of the physics principles of radiation attenuation in matter. In some implementations, first layer 306 is constructed of a low-atomic-number (“low-Z”) material, such as acrylic, which absorbs most of the energy of the incident electron beam while releasing minimum Bremsstrahlung radiation. In some implementations, second layer 308 is a high-atomic-number (“high-Z”) material, such as copper, which stops the remainder of the electrons and absorbs most of the low-energy Bremsstrahlung radiation emitted either in the first layer or the second layer itself. Stacking acrylic and copper, or other materials, in this way yields a greater reduction in radiation per unit weight added. In some implementations, first layer 306 has a relative thickness of 50% of second layer 308 at the expense of less than 1% dose increase. In some implementations, optional third layer 310 which again may be optional-is formed of a material with K-shell absorption energies that match the characteristic X-rays of the second material of second layer 308. This maximizes the amount of photon attenuation in optional third layer 310 per unit thickness. In some implementations, third layer 310 is a thin layer of metal.

When retained by mounting assembly 302, it will be appreciated that beam modifier 304 will be positioned at or near the exit window of linac 100. More specifically, beam modifier 304 will be positioned such that first layer 306 is nearer to the exit window than second layer 308. In other words, beam modifier 304 may be positioned such that the top surface of beam modifier 304 faces the exit window of linac 100 and the bottom surface of beam modifier 304 faces toward subject 330.

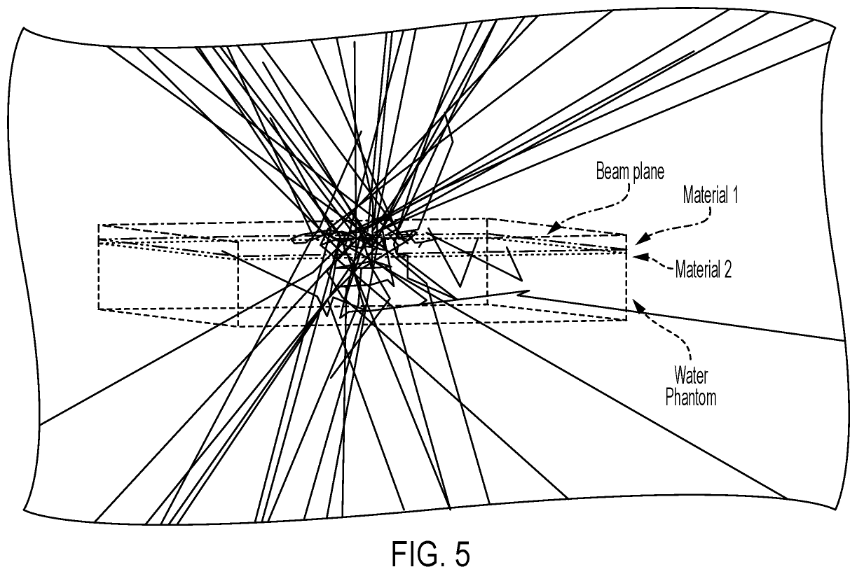

Referring now to FIG. 5, a diagram of simulation results for beam modifier 304 is shown, according to some implementations. In particular, Monte Carlo simulations were used to test the efficacy of beam modifier 304 given a 16 MeV beam spectrum. This methodology can be generalized to all electron energies as required. In the example geometry shown in FIG. 5, particle tracks are visualized, with green tracks being photons and red tracks being electrons. In the simulation, the electron beam plane lies immediately adjacent to the upstream surface of the beam modifier since it will be in contact with the applicator. The beam interacts with the modifier, shown here as a stack of two materials. To compare different modifier compositions, a water phantom was positioned downstream of beam modifier 304 which scores dose metrics.

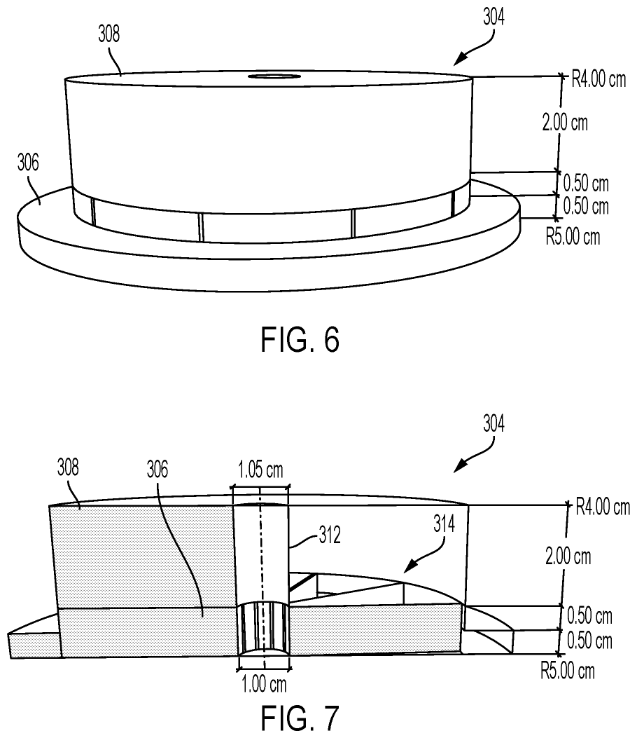

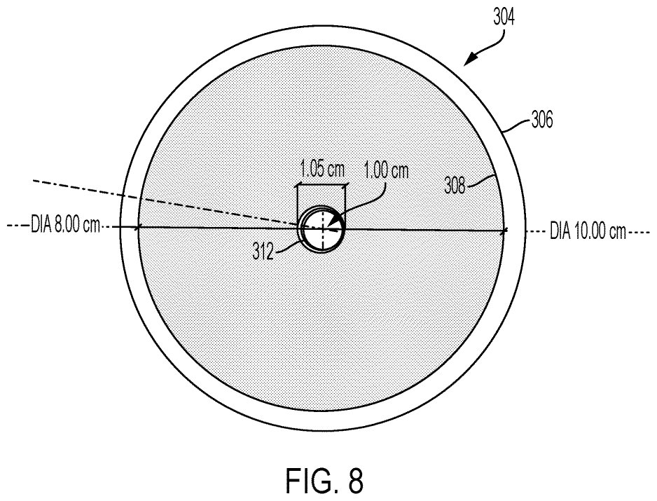

Referring now to FIGS. 6-8, various perspective views of a first configuration of beam modifier 304 are shown, according to some implementations. In this first configuration, beam modifier 304 is shown to be generally cylindrical in shape. Notably, first layer 306 is shown to form a flange that has a base having a first diameter, which is wider than a second of the body of beam modifier 304 as formed by second layer 308. In some implementations, the flange formed by first layer 306 interfaces with mounting assembly 302 to cause beam modifier 304 to be retained by mounting assembly 302. In some implementations, a central opening 312 is formed in beam modifier 304, which extends along a longitudinal axis of beam modifier 304 from a top surface to a bottom surface. In FIGS. 6-8, central opening 312 is shown to be generally cylindrical in shape. Accordingly, central opening 312 may be considered a central “bore.”

In some implementations, a plurality of fins 314 may be formed in first layer 306, as shown in FIG. 7, to reduce static buildup from the beam. Generally, fins 314 are formed of the same material as second layer 308. In one example, fins 314 are formed of copper. In some implementations, first layer 306 and second layer 308 are fabricated separately and later integrated into beam modifier 304. In some such implementations, first layer 306 may have a plurality of slots formed therein which are intended to align with fins 314. For example, slots may be milled into first layer 306. Likewise, fins 314 may be milled into or otherwise formed on second layer 308 and positioned to align with the slots in first layer 306 when the two portions of beam modifier 304 are brought together.

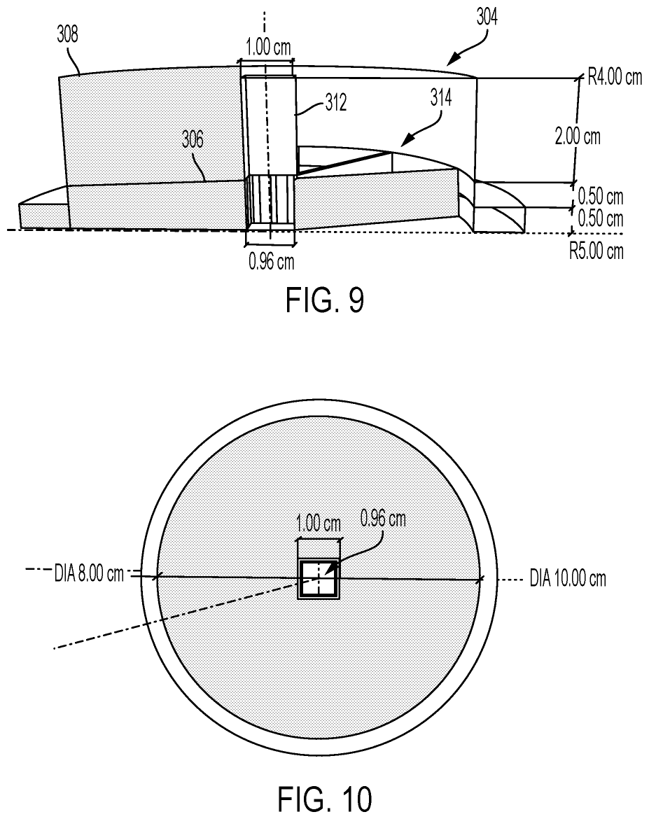

Referring now to FIGS. 9 and 10, various perspective views of a second configuration of beam modifier 304 are shown, according to some implementations. Broadly, the second configuration of beam modifier 304 is the same as or similar to the first configuration shown in FIGS. 6-8; however, as shown, central opening 312 is generally square or rectangular in shape, thus resulting in a different beam shape. It should be appreciated that any size or shape of opening can be formed into beam modifier 304 based on the desired irradiation field size/shape.

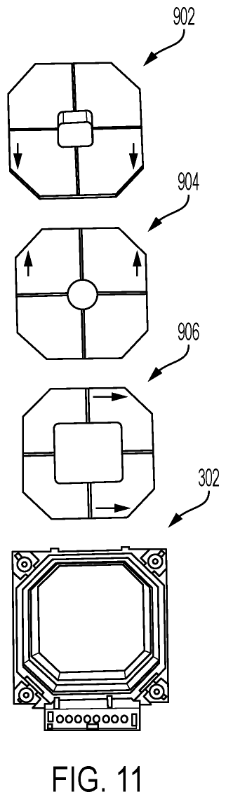

Referring now to FIG. 11, a diagram of an example implementation of beam-modifying assembly 300 is shown, according to some implementations. In this example, beam modifier 304 is shown as a plurality of differently configured cutouts 902-906, which may be formed of acrylic and copper, or other materials as mentioned above. As shown, each of cutouts 902-906 has a different size and/or shape of central opening (e.g., central opening 312). Each of cutouts 902-906 is shown to fit into mounting assembly 302—shown at the bottom of the figure. As described herein, mounting assembly 302 may be generally formed of any suitable material, such as plastic, metal, etc. In some implementations, mounting assembly 302 is 3D printed. Likewise, in some implementations, at least first layer 306 of beam modifier 304 can be 3D printed (e.g., of acrylic or other material). In some implementations, mounting assembly 302, first layer 306, and/or second layer 308 may be milled from a respective material. For example, in FIG. 11, cutouts 902-906 are shown to be milled from copper. As mentioned above, as another example, second layer 308 may be milled from copper or another material.

### Simulation Results

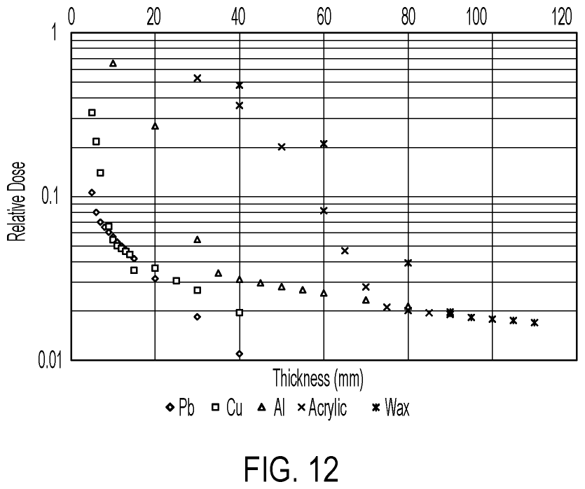

Referring now to FIG. 12, a graph illustrating results of simulating a beam-modifying device formed of a single material is shown, according to some implementations. In this example, the relative dose to a water phantom, positioned downstream of the exit window of linac 100, is plotted on a log scale against the thickness of a single-material (e.g., single-layer) beam modifier, and normalized by the dose observed when no beam modifier is present. Higher Z materials, such as lead, have curves that are lower and to the left since they have higher mass stopping power than low-Z materials. A sharp change in each curve is observable where the electron beam is fully absorbed, and Bremsstrahlung becomes the greatest contributor. This inverse shoulder occurs at a higher relative dose for high-Z materials since they have a higher efficiency of Bremsstrahlung production.

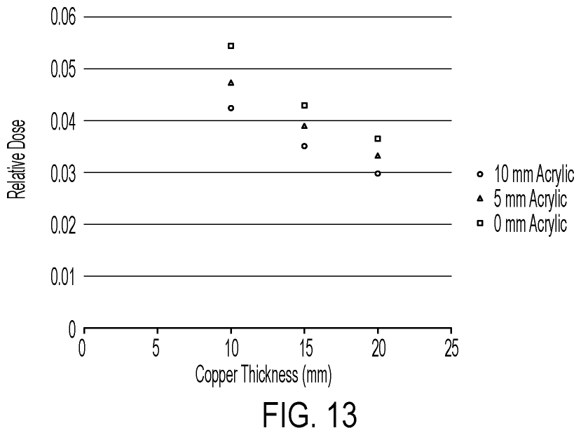

Referring now to FIG. 13, a graph illustrating results of simulating a beam-modifying device formed of multiple different materials is shown, according to some implementations. This plot shows the relative water phantom dose with beam modifier 304 composed of acrylic on top of copper. Designs with three acrylic layer thicknesses and three copper layer thicknesses are also shown. Naturally, it can be seen that a thicker beam modifier results in a lower dose. However, it is also shown that a modifier of the same total thickness can be made with a larger proportion of acrylic and have comparable dose reduction. Of note, a 10 mm Cu, 10 mm acrylic beam modifier is within 0.5% of a 15 mm Cu, 5 mm acrylic. An even smaller difference is observable when comparing the 15 mm Cu, 5 mm acrylic beam modifier to a 20 mm Cu, 0 mm acrylic beam modifier. This non-linearity is a result of lower Bremsstrahlung production in the acrylic, which allows the acrylic layer to decrease the energy of the electron beam while producing fewer high energy photons.

### Configuration of Certain Implementations

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

The construction and arrangement of the systems and methods as shown in the various implementations are illustrative only. Although only a few implementations have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

What is claimed is:

1. A beam-modifying assembly for an ultra-high dose rate (UHDR) electron linear accelerator, the beam-modifying assembly comprising:
a beam modifier formed of a first material that defines a first layer and a second material that defines a second layer, the second material being different from the first material, wherein the first layer is provided on top of the second layer such that a first surface of the beam modifier comprises the first material and a second surface of the beam modifier comprises the second material, the second surface being on an opposite side of the beam modifier from the first surface; and
a mounting assembly configured to retain the beam modifier, wherein the mounting assembly is adapted to be installed at an exit window of the linear accelerator such that the beam modifier is positioned at the exit window of the linear accelerator, wherein the first surface of the beam modifier faces the exit window and the second surface faces a subject to be irradiated.

2. The beam-modifying assembly of claim 1, wherein the first material is a low-atomic-number (low-Z) material and the second material is a high-atomic-number (high-Z) material.

3. The beam-modifying assembly of claim 1, wherein the first material is acrylic.

4. The beam-modifying assembly of claim 1, wherein the second material is copper.

5. The beam-modifying assembly of claim 1, wherein the beam modifier is cylindrical in shape.

6. The beam-modifying assembly of claim 5, wherein the second layer forms a flange, and wherein a base of the flange interfaces with the mounting assembly to retain the beam modifier in the mounting assembly.

7. The beam-modifying assembly of claim 5, wherein the first layer of the beam modifier is formed in a cylindrical shape defined by a first diameter, and wherein the second layer is formed in the shape of a flange having a base diameter that is wider than the first diameter.

8. The beam-modifying assembly of claim 1, further comprising a third layer formed of a third material that is different from the first material and the second material, wherein the third layer is provided on a bottom surface of the second layer such that the second surface of the beam modifier comprises the third material.

9. The beam-modifying assembly of claim 8, wherein the third material is a metal having K-shell absorption energies that match characteristic X-rays of the second material.

10. The beam-modifying assembly of claim 1, wherein the beam modifier is 3D printed, cast, or milled from the first material and the second material.

11. The beam-modifying assembly of claim 1, wherein a central opening is formed in the beam modifier, the central opening extending from the first surface to the second surface along a longitudinal axis of the beam modifier.

12. The beam-modifying assembly of claim 11, wherein the central opening is cylindrical in shape.

13. The beam-modifying assembly of claim 11, wherein the central opening is square in shape.

14. The beam-modifying assembly of claim 1, wherein the first layer has a relative thickness that is 50% of a thickness of the second layer.

15. The beam-modifying assembly of claim 1, wherein beam modifier further comprises a plurality of fins that extend from the first layer and into the second layer, wherein the plurality of fins are formed of the first material.
